SEQUENCE LISTING (1 sequence)

SEQ ID NO: 1 - protein, 39 aa, Homo sapiens
SYSMEHFRWGKPVGKKRRPVKVYPNGAEDESAEAFPLEF

(19) United States
(12) Patent Application Publication
(10) Pub. No.: US 2016/0123989 A1
(43) Pub. Date: May 5, 2016

(54) METHODS AND KITS OF DETERMINING WHETHER A SUBJECT IS SUSCEPTIBLE TO HAVE A HYPER-AGGRESSIVE BEHAVIOUR

(71) Applicants: INSERM (INSTITUT NATIONAL DE LA SANTÉ ET DE LA RECHERCHE MÉDICALE), Paris (FR); UNIVERSITÉ DE ROUEN, Mont-Saint-Aignan (FR); CENTRE HOSPITALIER UNIVERSITAIRE DE ROUEN, Rouen (FR); AKERSHUS UNIVERSITETSSYKEHUS HF, Lørenskog (NO)

(72) Inventors: Serguei FETISSOV, Rouen (FR); Pierre DECHELOTTE, Rouen (FR); Henning VÆRØY, Lørenskog (NO); Romain LEGRAND, Rouen (FR)

(21) Appl. No.: 14/898,628

(22) PCT Filed: Jun. 20, 2014
(86) PCT No.: PCT/EP2014/063062

§ 371 Date: Dec. 15, 2015

(30) Foreign Application Priority Data
Jun. 21, 2013 (EP) 13305850.3

(51) Int. Cl. G01N 33/68

(52) U.S. Cl. CPC G01N 33/6854; G01N 2333/695

(57) ABSTRACT

The present invention relates to methods and kits of determining whether a subject is susceptible to have a hyper-aggressive behaviour. In particular, the present invention relates to a method for determining whether a subject is susceptible to have a hyper-aggressive behaviour comprising the steps consisting of i) isolating the auto antibodies that bind to adrenocorticotropic hormone (ACTH) from a blood sample obtained from the subject, ii) determining the affinity of the isolated autoantibodies iii) comparing the affinity determined at step ii) with a predetermined reference value and iv) concluding that the subject is susceptible to have a hyper-aggressive behaviour when the affinity determined at step ii) is higher than the predetermined reference value.

### FIELD OF THE INVENTION

The present invention relates to methods and kits of determining whether a subject is susceptible to have a hyper-aggressive behaviour.

### BACKGROUND OF THE INVENTION

Current approaches in understanding the mechanism of conduct disorder (CD) include genetic, biochemical, environmental, and social factors, but to what extent these factors, alone or in combination, contribute to a common pathogenesis of aggressive and antisocial behavior remains uncertain. One strategy to study CD could be to view its aggressive component as an inappropriate expression of motivated behaviors that include eating, drinking, reproductive, defensive-aggressive, and explorative behaviors as well as sleep and thermoregulation that normally serve to maintain body homeostasis. Progress in animal studies show that selective, mainly limbic neural circuitries generate urges to be expressed as motivated behaviors leading, when accomplished, to feeling of pleasure and to the reduced activity of the hypothalamic-pituitary-adrenal (HPA) axis. Several neuropeptides such as corticotropin (ACTH), α-melanocyte-stimulating hormone (α-MSH), oxytocin (OT), and vasopressin (VP) play important roles as neurohormones or messengers in regulation of the HPA axis activity and in regulation of brain mechanisms of various motivated behaviors including defensive/aggressive behavior. Moreover, HPA activity, via the secretion of cortisol, appears to be highly relevant to the mechanisms of human aggression, including conduct disorder and antisocial personality disorder. It was reported that high levels of ACTH-reactive autoantibodies as well as altered levels of oxytocin- and vasopressin-reactive autoantibodies found in aggressive subjects may interfere with the neuroendocrine mechanisms of stress and motivated behavior. These data suggest a new biological mechanism of human aggressive behavior that involves autoantibodies directed against several stress-related neurohormones.

### SUMMARY OF THE INVENTION

The present invention relates to methods and kits of determining whether a subject is susceptible to have a hyper-aggressive behaviour.

### DETAILED DESCRIPTION OF THE INVENTION

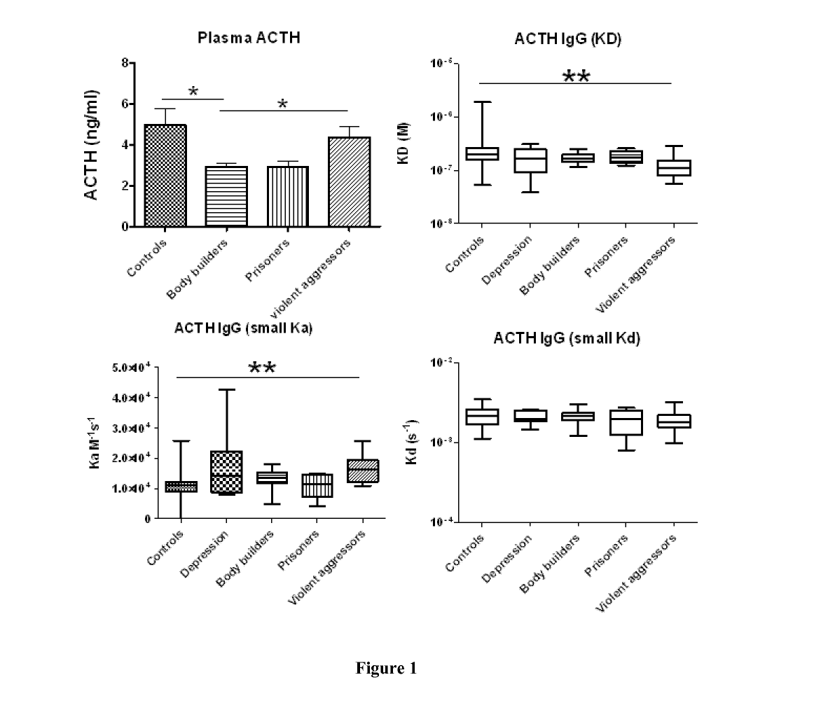

The inventors introduce a hypothesis proposing a neurobiological model for understanding some of the mechanisms behind extreme aggression via over activation of the stress axis mediated by plasma immunoglobulins (Ig). Their main study group was male prison inmates serving long time sentences, the majority in preventive detention. They all had confirmed hyper-aggressive behaviour in form of having committed murder or other extreme forms of violence against others. Inmates serving time for sexual deviances such as paedophilia were not included. For comparison, the inventors used four male control groups: 1)—a group of healthy non-criminal volunteers, 2)—a group of prisoners serving short time sentences for less aggressive behaviour, 3)—a group of body builders actively using a range of performance enhancing substances (steroids) and 4)—a group of severely depressed patients admitted for electroconvulsive treatment. They were all tested for plasma ACTH peptide levels and for affinity kinetics of plasma IgG for ACTH using surface plasmon resonance phenomenon on a BIAcore instrument. The plasma concentration of the ACTH peptide did not differ significantly when comparing the levels found in the study groups included. However, IgG purified from plasma from the group of violent aggressors displayed significantly higher micromolar affinity for ACTH (lower KD, due to increased Ka) than did the healthy non-criminal controls (ANOVA p=0.003, Dunn's p<0.01) (FIG. 1). One way of interpreting these findings is that the higher micromolar affinity IgG might be responsible for more efficient ACTH transport and thus may be one of the biological reasons for the hyperarousal type of aggression.

Accordingly the present invention relates to a method for determining whether a subject is susceptible to have a hyper-aggressive behaviour comprising the steps consisting of i) isolating the autoantibodies that bind to adrenocorticotropic hormone (ACTH) from a blood sample obtained from the subject), ii) determining the affinity of the isolated autoantibodies iii) comparing the affinity determined at step ii) with a predetermined reference value and iv) concluding that the subject is susceptible to have a hyper-aggressive behaviour when the affinity determined at step ii) is higher than the predetermined reference value.

As used herein the term “ACTH” has its general meaning in the art and refers to the adrenocorticotropic hormone, also known as corticotropin. ACTH is 39 amino acid polypeptide hormone produced from its precursor proopiomelanocortin and secreted by the anterior pituitary gland. It is an important component of the hypothalamic-pituitary-adrenal axis and its increased secretion is typically occurs in response to biological and psychological stress (along with ACTH releasing factor, corticotropin-releasing hormone (CRH) from the hypothalamus). Its principal effects are increased production and release of corticosteroids from the adrenal cortex. An exemplary human amino acid sequence of ACTH is SEQ ID NO:1.

SEQ ID NO: 1:
SYSMEHFRWGKPVGKKRRPVKVYPNGAEDESAEAFPLEF

An “autoantibody” has its general meaning in the art and refers to the natural immunoglobulins (Ig) of the subject. Antibodies consists of two heavy chains are linked to each other by disulfide bonds and each heavy chain is linked to a light chain by a disulfide bond. There are two types of light chain, lambda (l) and kappa (k). There are five main heavy chain classes (or isotopes) which determine the functional activity of an antibody molecule: IgM, IgD, IgG, IgA and IgE. The term “binding” of a compound or ligand occurs by intermolecular forces, such as ionic bonds, hydrogen bonds and van der Waals forces. The docking (association) between two compounds such as a ligand and its target molecule is reversible in biological systems. In a particular embodiment, the isolated autoantibodies of the subject are of the IgG class.

As used herein the term “blood sample” has its general meaning in the art and refers to a whole blood sample, a plasma sample or a serum sample.

Techniques for isolating autoantibodies from a blood sample are well known in the art. For example, total IgG are typically purified from the plasma sample that was previously acidified for peptide extraction. The IgG purification may be performed using Melon Gel Kit (cat. N. 45206) according to the manufacturer instructions (Thermo Scientific, Pierce, Rockford, USA). IgG containing effluents can be saved and frozen at −20° C. before determining the affinity of the autoantibodies.

Typically affinity of an antibody can be described by kinetic rates and affinity constants. The “kinetics” of a reaction describes the time dependency of the binding event. Therefore, it comprises information about i) how fast a compound binds to or associates with another compound, wherein the velocity of that reaction is described with an association rate constant (kₐ); ii) and how fast a compound dissociates from another compound, wherein the velocity of that reaction is described with the dissociation rate constant (k_{d}). The equation for two compounds (A and B) binding each other (AB) is given by the equation (1):

$$A + B {}^{{k}_{a}} {}_{{K}_{d}} AB \qquad ( 1 )$$

The “rate of association” of said reaction is the number of binding events per unit of time and equals [A]*[B]*kₐ, wherein kₐ is the “association rate constant” (kₐ) also known in literature as <<kₒₙ>> with the unit M⁻¹s⁻¹. Once binding has occurred, the compounds remain bound together for a random amount of time. The “rate of dissociation” is the number of dissociation events per unit time and equals [AB]*k_{d}. The probability of dissociation is the same at every instant of time. Equilibrium is reached when the rate of formation of new AB complexes equals the rate at which existing AB complexes dissociate. By definition the equilibrium can be defined by equation (2):

[A]*[B]*kₐ=[AB]*K_{d} (2)

This equation can be rearranged to define another constant describing the equilibrium of the reaction nominated as the equilibrium dissociation constant (K_{D}). The equilibrium dissociation constant (K_{D}) depends on temperature and partial pressure of the reaction mixture comprising the two compounds A and B is defined as:

$$\frac{[ A ] * [ B ]}{[ AB ]} = \frac{{K}_{d}}{{k}_{a}}$$

The smaller the dissociation constant, the more tightly bound the compound is, or the higher the affinity between the two compounds. For example, a ligand with a nanomolar (nM) dissociation constant binds more tightly to a particular receptor than a ligand with a micromolar (μM) dissociation constant.

The affinity kinetics can be measured for example by a multi-cycle method on the surface plasmon resonance (SPR) phenomenon on a BIAcore Upgrade instrument (BIAcore, GE Healthcare, Piscataway, N.J. In one example, acyl-ACTH is diluted in a sodium acetate buffer, pH 5.0 (BIAcore) and was covalently coupled on the sensor chip CM5 (BIAcore), using the amine coupling kit (BIAcore). The multi-cycle method may be run for example with five serial dilutions of each patient and control IgG: 0.5, 0.25, 0.125, 0.625 and 0.03125 (mg/mL) including a duplicate of 0.125 mg/ml and a blank (buffer only). During each cycle, an amount is injected into the flow conduit of the BIAcore instrument with flow speed 30 μl/min at 25° C. and 5 min of dissociation. Between injections of each sample, the binding surface was regenerated with NaOH. The affinity kinetic data can be analysed using BiaEvaluation 4.1.1 program (BIAcore). For fitting kinetic data, the Langmuir's 1:1 model may be used and the sample values are corrected by subtracting the blank values resulting from the injection of HBS-EP buffer.

The inventors found that autoantibodies binding to ACTH in subjects susceptible to have a hyper-aggressive behaviour have an affinity to ACTH in the micromolar range (μM). The inventors hypothesize the autoantibodies mediate the transport of the autoantibodies because they will not compete with the nano- to pico-molar affinity of interaction between ACTH and its receptor. The role of the ACTH-reactive IgG in the individuals may be the protection of ACTH from degradation. Thus, the slight increase in affinity of ACTH-reactive IgG found in subject susceptible to have a hyper-aggressive behaviour might improve their properties as ACTH carriers/protectors, while not antagonizing ACTH receptor binding.

In a particular embodiment, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the nucleic acid levels (obtained according to the method of the invention) with a defined threshold value. In one embodiment of the present invention, the threshold value is derived from the affinity determined in healthy population (e.g. a healthy non criminal subjects). Furthermore, retrospective measurement of the affinity properly banked historical of subjects may be used in establishing these threshold values.

In some embodiments the method of the invention comprises the step consisting of determining the KD of the autoantibodies ii) comparing the KD determined at step i) with a predetermined reference value and iii) concluding that the subject is susceptible to have a hyper aggressive behavior when the KD determined at step i) is lower than the predetermined reference value.

In some embodiments the method of the invention comprises the step consisting of determining the Ka of the autoantibodies ii) comparing the Ka determined at step i) with a predetermined reference value and iii) concluding that the subject is susceptible to have a hyper aggressive behavior when the Ka determined at step i) is higher than the predetermined reference value.

The method of the invention may be useful for prescribing the accurate psychological and other treatments for the subject. For example, subjects that are considered as to be susceptible to have a hyper-aggressive behaviour may be placed in conditions for limiting their stress and may administrated with a therapeutic treatment that will limit the aggressive manifestations. Such a method may thus help the physician to make a choice on a therapeutic treatment. Costs of the treatments may therefore be adapted to risk of the subjects.

The present invention also relates to a kit for implementing the method of the invention comprising means for determining the affinity of autoantibodies for ACTH. In some embodiments, the kit may also comprise means for isolating the antibodies from a blood sample.

### REFERENCES

Throughout this application, various references describe the state of the art to which this invention pertains. The disclosures of these references are hereby incorporated by reference into the present disclosure.

1. A method for determining whether a subject is susceptible to have a hyper-aggressive behaviour comprising the steps of i) isolating the autoantibodies that bind to adrenocorticotropic hormone (ACTH) from a blood sample obtained from the subject, ii) determining the affinity of the isolated autoantibodies, iii) comparing the affinity determined at step ii) with a predetermined reference value and iv) concluding that the subject is susceptible to have a hyper-aggressive behaviour when the affinity determined at step ii) is higher than the predetermined reference value.

2. The method of claim 1 which comprises the step of i) determining the KD of the autoantibodies ii) comparing the KD determined at step i) with a predetermined reference value and iii) concluding that the subject is susceptible to have a hyper aggressive behavior when the KD determined at step i) is lower than the predetermined reference value.

3. The method of claim 1 which comprises the step of i) determining the Ka of the autoantibodies ii) comparing the Ka determined at step i) with a predetermined reference value and iii) concluding that the subject is susceptible to have a hyper aggressive behavior when the Ka determined at step i) is higher than the predetermined reference value.

4. The method of claim 1 wherein the affinity kinetics is measured by surface plasmon resonance (SPR).

5. The method of claim 1 wherein the predetermined reference value is a threshold value derived from the affinity determined in healthy non-criminal subjects.
